(19) United States
(12) Patent Application Publication
(10) Pub. No.: US 2015/0094631 A1
(43) Pub. Date: Apr. 2, 2015

(54) KNEE BRACE SUPPORT

(71) Applicant: MARLENA INTERNATIONAL, LLC, Lawrenceville, GA (US)

(72) Inventor: Vera A. WATKINS, Lawrenceville, GA (US)

(21) Appl. No.: 14/504,928

(22) Filed: Oct. 2, 2014

Related U.S. Application Data
(60) Provisional application No. 61/885,910, filed on Oct. 2, 2013.

(51) Int. Cl. A61F 5/01

(52) U.S. Cl. CPC A61F 5/0123; A61F 2005/0172
USPC 602/16; 602/26

(57) ABSTRACT

A support article for maintaining a knee brace in alignment with a wearer's knee, comprising a first leg panel and a second leg panel, at least one of the leg panels including upper and lower outside pockets, and upper and lower inside pockets, the pockets being positioned generally on the side area of a panel. Each pocket has an opening and is adapted to hold a portion of a knee brace support arm. A strap is associated with each pocket, each strap being adjustable. A zipper may extend from a bottom opening of a leg panel toward a thigh area. The support article maintains the knee brace in position proximate to the knee area, so that the knee brace does not appreciably slide down the wearer's leg.

### CROSS-REFERENCE TO RELATED APPLICATION

This patent application claims priority to U.S. Provisional Patent Application No. 61/885,910, filed Oct. 2, 2013, the disclosure of which is incorporated herein by reference in its entirety.

### FIELD

The present disclosure relates, in exemplary embodiments, to articles for providing support to a user of a knee or leg brace.

### SUMMARY

In exemplary embodiments, a support article or garment for supporting and maintaining at least one knee brace in position proximate to a knee is provided. The knee brace may comprise a pair of support arms connected to a hinge. The garment may comprise a first leg panel having a thigh portion, a knee portion and a calf portion; upper and lower outside pockets associated with the first leg panel, each pocket sized to accept at least a portion of a support arm; upper and lower inside pockets associated with the first leg panel, each pocket sized to accept at least a portion of a support arm and, a plurality of strap members, each strap member having a first end and a second end adjustably connectable by an adjustment connector, each strap member being associated with a portion of a pocket.

In exemplary embodiments, the second panel has a thigh portion, a knee portion and a calf portion; upper and lower outside pockets associated with the second leg panel, each pocket sized to accept at least a portion of a support arm; upper and lower inside pockets associated with the second leg panel, each pocket sized to accept at least a portion of a support arm and, a plurality of strap members, each strap member having a first end and a second end adjustably connectable by an adjustment connector, each strap member being associated with a portion of a pocket.

In exemplary embodiments, a garment for supporting and maintaining at least one knee brace in position proximate to a knee is provided. The knee brace may comprise a pair of support arms connected to a hinge. The garment may comprise a first leg panel having a thigh portion, a knee portion and a calf portion; upper and lower outside pockets associated with the first leg panel, each pocket sized to accept at least a portion of a support arm; upper and lower inside pockets associated with the first leg panel, each pocket sized to accept at least a portion of a support arm and, a plurality of strap members, each strap member having a first end and a second end adjustably connectable by an adjustment connector, each strap member being associated with a portion of a pocket. The garment further includes a second leg panel having a thigh portion, a knee portion and a calf portion; upper and lower outside pockets associated with the first leg panel, each pocket sized to accept at least a portion of a support arm; upper and lower inside pockets associated with the first leg panel, each pocket sized to accept at least a portion of a support arm and, a plurality of strap members, each strap member having a first end and a second end adjustably connectable by an adjustment connector, each strap member being associated with a portion of a pocket.

In exemplary embodiments, a knee brace support is provided, comprising a fabric panel having a first side edge, a second side edge, a top edge, and a bottom edge; at least one row of first closure devices associated with an area proximate to the first side edge; at least one row of second closure devices associated with an area proximate to the second side edge, the second closure devices adapted to fasten to the first closure devices; and, at least one pocket associated with the fabric panel, each pocket having an opening. In exemplary embodiments, the knee brace support further comprises at least one support arm removably retained in each pocket.

### BRIEF DESCRIPTION OF THE DRAWINGS

The drawings disclose exemplary embodiments in which like reference characters designate the same or similar parts throughout the figures of which:

FIG. 1 is a front perspective schematic view of one exemplary embodiment of article support garment according to an exemplary embodiment of the present disclosure.

FIG. 2 is a side perspective schematic view of the exemplary embodiment of FIG. 1.

FIG. 3 is a rear schematic view of the exemplary embodiment of FIG. 1.

FIG. 4 is a rear schematic view of a support garment having first and second leg portions that are each able to secure a knee brace, according to one exemplary embodiment.

FIG. 5 is a front schematic view of a support according to an exemplary embodiment.

FIG. 6 is a rear schematic view of the support of FIG. 5.

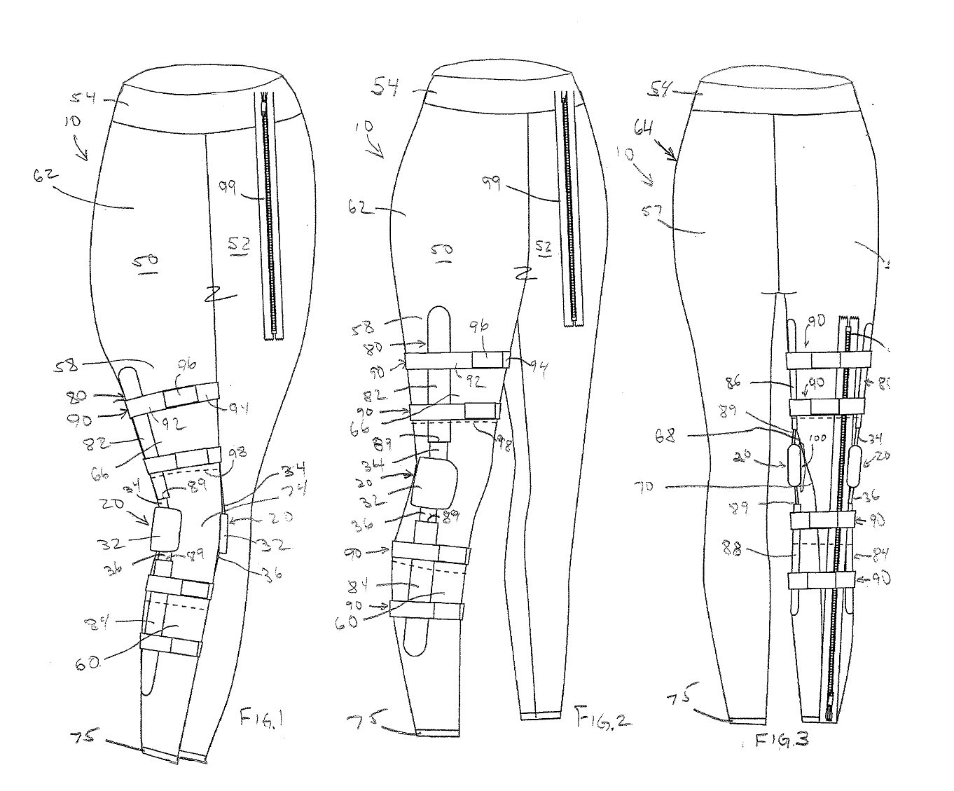

### DETAILED DESCRIPTION

In exemplary embodiments, a support garment 10 is provided for securing a knee brace 20 to a user's leg 30. A typical knee brace 20 has a hinge 32 and a pair of support arms 34, 36 that are designed to be strapped or otherwise secured to the thigh area and calf area of a user.

In exemplary embodiments, a support garment 10 may comprise a first leg panel 50. In exemplary embodiments, a support garment 10 may also include a second leg panel 52 and a waist portion 54. In exemplary embodiments, the first and second leg panels 50, 52 each include a seat portion 56, 57, an upper leg portion 58 and a lower leg portion 60, respectively, and front and back portions 62, 64. In exemplary embodiments, at least a portion of the support garment 10 may be made of a material having at least a portion thereof made of a fabric that is elastic or that provides a degree of compression or constriction. Such fabrics are commonly known as compression fabrics and have elasticity that provides pressure to the outside of a body area. Such fabrics are commonly used after surgery to maintain a compressive pressure on the body area, which may help promote post-operation healing, reduce swelling and the chance of edema. An example of such compression fabric is COMFORTWEAVE® fabric (available from Marena Group, Lawrenceville, Ga.). In exemplary embodiments, the entire fabric can be made of the same fabric with the same compression elasticity throughout the fabric. Alternatively, the support garment 10 may have certain areas made of fabric with higher levels of compression. In exemplary embodiments, areas proximate to the knee may be made so as to have higher compression as such areas may be prone to swelling after knee surgery than the surrounding area above or below the knee.

The first leg panel 50 includes an outside area 66 and an inside area 68. In exemplary embodiments, the first leg panel 50 may have a rear seam 70 (see FIG. 3) that starts at the crotch as being on the inside area 68 and curves proximate to the back of the knee so that the seam 70 is not located in an area that is directly between the knee brace elbow and the skin, thus reducing potential rubbing and irritation of the seam during use. In exemplary embodiments, the first leg panel 50 has a thigh portion 58, knee portion 74 and calf portion 60 generally corresponding to those body areas of a user. The first leg panel 50 also has a bottom opening 75.

In exemplary embodiments, the first leg panel 50 includes a zipper 78 extending from the bottom opening 75 to generally above the knee. In exemplary embodiments, the zipper 78 may extend to below the knee or above the knee toward the thigh area. In exemplary embodiments, the zipper 78 is positioned on the back side of the first leg panel 50, or may be positioned between the back area and the outside of the first leg panel 50. The zipper 78 can facilitate the user removing a leg from or inserting a leg into the first leg panel 50, particularly where the user's leg likely has restricted movement and pain post-surgery.

In exemplary embodiments, one or more sleeve pockets (generally referred to as 80) are included in the support garment 10. In exemplary embodiments, the first leg panel outside area 66 includes an upper leg sleeve pocket 82 and a lower leg sleeve pocket 84, and the inside area includes an upper leg sleeve pocket 86 and a lower leg sleeve pocket 88. Each sleeve pocket 80 is attached to or formed as part of the first leg panel 50. Each sleeve pocket 80 has an opening 89 sized to accommodate a leg brace support arm 34 or 36. The length of each sleeve pocket 80 is generally sufficient to accommodate at least a portion of the length of the support arm 34, 36. In exemplary embodiments, one knee brace 20 is used on each side of the knee area. In other exemplary embodiments, only one knee brace 20 is used on either side of the knee, such as where the user requires less bracing or support. In exemplary embodiments, where one knee brace 20 is used, the support garment may be constructed to have just one pair of upper and lower leg sleeve pockets 80, either just on the outside area 66 or just on the inside area 68. The sleeve pockets 80 maintain the support arms 34, 36 in-line with the leg 30.

In exemplary embodiments, at least one securing strap 90 is used to maintain the brace support arms 34, 36 in position. In exemplary embodiments, as shown in the figures, two upper leg straps 90 and two lower leg straps 90 are utilized. Each strap 90 may include, in exemplary embodiments, a first segment 92, a second segment 94, and at least one adjustment connector 96. In exemplary embodiments, the strap 90 may be constructed of hook and loop material. In exemplary embodiments, the first and second strap segments 92, 94 may be made of the same side of the hook and loop material, i.e., both made of the hook material or both made of the loop material. In exemplary embodiments, the adjustment connector 96 is made of the other, mating material. In exemplary embodiments, two adjustment connectors 90 are used, one joining the first and second strap segments 92, 94 in the front of the first leg panel 50, and a second adjustment connector 96 joining the first and second segments 92, 94 in the first leg panel back portion 64. Each adjustment connector 96 allows for adjustably joining the first and second segments 92, 94 of the particular strap 90, which may be advantageous as different users have calves or thighs with a different thickness, and, such areas may be thinner later after surgery than immediately after the surgery.

In exemplary embodiments, one or more of the straps 90 may be removably attachable to a portion of one or more sleeve pockets 80. In exemplary embodiments, each sleeve pocket 80 may be made of (or have a portion made of) one side of a hook and loop material, such as the loop side, and each strap 90 may have a portion of the material be constructed of or associated with the mating material, such as the hook side, such that the strap 90 may attach to the sleeve pocket 80. In alternative exemplary embodiments, the strap 90 may be attachable to or associated with the sleeve pocket 80 via a loop associated with the sleeve pocket 80 that permits movement of the strap 90 within the loop, thus preventing appreciable movement up or down the user's leg, but permits rotational movement of the strap around the leg. Such construction in these various exemplary embodiments can prevent the strap 90 from moving up or down on the leg 30 when the support garment 10 is used. The detachable straps 90 enable the support garment 10 to be washed. A user can remove the straps 90, and optionally also remove the knee brace 20, so that the support garment 10 can be worn without the knee brace 20, such as at night, when compression may be desirable, but there is no need for the knee brace 20.

The width of all the straps 90 can be the same, or different width straps 90 may be used. In exemplary embodiments, the width of the first and second strap segments 92, 94 and the adjustment connector 96 is the same. In other exemplary embodiments, the width of at least a portion of a strap segment 92 or 94 may vary, such as, but not limited to, by being narrower in the front and wider in the back, or vice versa. In exemplary embodiments, a narrower width strap 90 may provide a more even fit, particularly around a thigh or calf area that is relatively tapered, such as where the calf muscles are very developed. In exemplary embodiments, a wider width strap 90 may provide more comfort where the thigh or calf area is softer and more uniform in diameter so as to spread out the strap pressure.

In other exemplary embodiments, the strap 90 may be constructed of fabric or other flexible material and utilize a closure mechanism, such as, but not limited to, hooks and eyes, buttons, metal or plastic loops, snaps, pins, or the like. In such embodiments, the strap 90 may be removably attachable or permanently affixed to the sleeve pocket 80 or the fabric as described above. In exemplary embodiments, at least a portion of the strap 90 may be formed of an elastic material.

In exemplary embodiments, the first leg panel 50 may further include a reinforcement layer 98 comprising a second layer of fabric. The reinforcement layer 98 may encircle or partially encircle the leg 30 in the area from above to below the knee. In exemplary embodiments, the reinforcement layer 98 is formed as part of the first leg panel 50 or attached thereto. In exemplary embodiments, the reinforcement layer 98 may be a separate sleeve that is slid over the knee area prior to inserting the leg into the first leg panel 50. In exemplary embodiments, the reinforcement layer 98 may be one or more smaller panels that are placed at various points around the knee area; each reinforcement layer panel 98 may encircle a portion of the leg, or may be of a patch-like size. The reinforcement layer 98 can provide additional compression force to the general area around the knee. The reinforcement layer 98 can also provide increased cushioning protection between the knee brace 20 and the skin to reduce the possibility of irritation due to rubbing or chafing of the knee brace support arms 34, 36 or the hinge 32.

The second leg panel 52 may have a zipper 99 positioned at the waist portion and extending at least part way down the leg to facilitate donning of a garment.

In exemplary embodiments, one or more cushioning pads 100 may be incorporated in either or both panels. In exemplary embodiments, one or more cushioning pads 100 may be incorporated on one or both sides of the knee area to provide additional cushioning between the skin and the hinge 32. In exemplary embodiments, the cushioning pad 100 may comprise a compressible material, such as, but not limited to, foam, rubber, silicone, plastic or other material.

In exemplary embodiments, the support garment 10 can be manufactured on a made-to-measure basis.

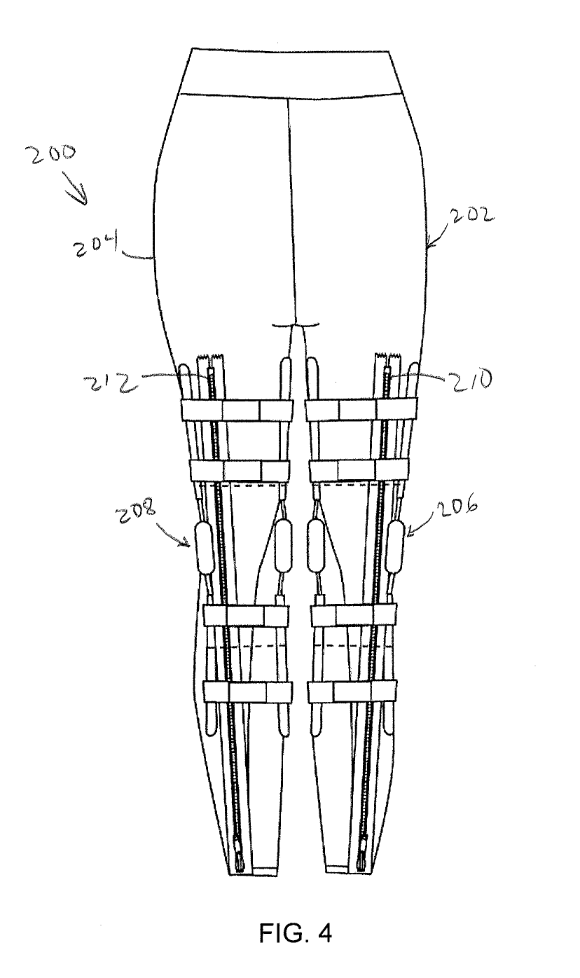

In exemplary embodiments, illustrated in FIG. 4, a support garment 200 can be constructed to have a first panel 202 constructed similar to the first leg panel 50 and may also have a second leg panel 204 include support arm sleeves and straps similar to those described hereinabove with respect to the first leg panel 50 (but as a mirror image) such that the first panel 202 accommodates a first knee brace 206 and the second panel accommodates a second knee brace 208. The support garment 200 may also include a first leg portion zipper 210 and a second leg portion zipper 212. This support garment 200 can be used by patients who have had surgery on both knees. A feature of the support garment of the present disclosure is that the support garment maintains the knee brace 20 in position alongside the knee area and reduces the likelihood of the knee brace 20 sliding down the leg due to movement and gravity. Another feature of the support garment is that it enables a user to easily slide the leg panel(s) over the injured/treated knee, which is usually very sensitive to bending and pressure.

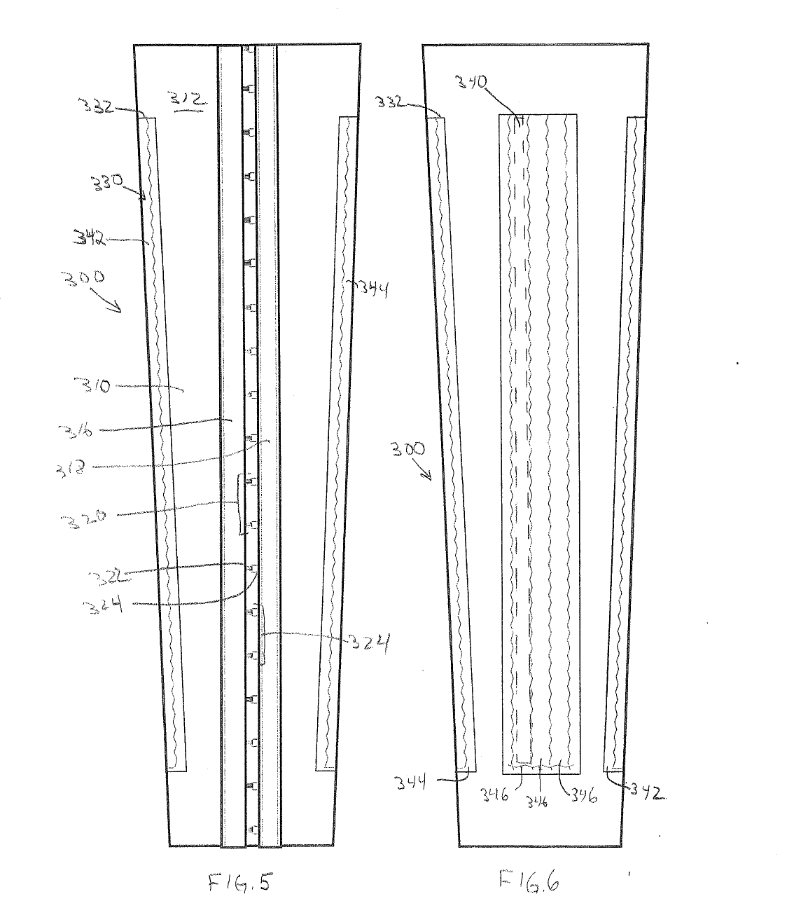

In exemplary embodiments, as shown in FIGS. 5-6, a knee brace support garment 300 is provided that comprises a leg support panel 310, at least a portion of which is made of an elastic fabric or compression fabric, as described hereinabove. The support panel 310 has an inside and an outside 312, 314. The support panel has a first side edge 316 and a second side edge 318. The first side edge 316 may have at least one row 320 of first closure devices 322, such as hooks, and the second side edge 318 having at least one row 324 of mating second closure devices 326, such as eyes. It is to be understood that other closure devices known to those of ordinary skill in the art may be used, such as, but not limited to, snaps, buttons, tabs, hook and loop fasteners, or the like. In exemplary embodiments, the first side edge 316 may have several rows 320 of first closure devices 322 so that the user can select which row 320 of first closure devices 322 to use based on the thickness of the user's leg. Selecting a row 320 farther away from the first side edge 316 will result in the support garment 300, when fitted on the user, having a smaller diameter. It is to be understood that the rows 320 of closure devices are not necessary all parallel, but may be angled, flared or tapered. It is also to be understood that the support panel 310 may have the second side edge 318 include several rows of second closure devices 326, rather than the first side edge 316. In alternative exemplary embodiments, both side edges 316, 318 may have several rows of closure devices. It is to be understood that the first closure devices 322 and second closure devices 326 can be switched.

In exemplary embodiments, the support panel 310 includes at least one elongated pocket 330. In exemplary embodiments, the support panel 310 includes a plurality of elongated pockets 330. In exemplary embodiments, at least a portion of each pocket 330 is made of a compression, stretch or elastic fabric as described hereinabove. In exemplary embodiments, each pocket 330 has an open end 332. In exemplary embodiments, each pocket 330 may be sewn onto or formed as part of the support panel 310. In exemplary embodiments, the pockets 330 may be placed all on the outside 312 or on the inside 314 of the support panel 310. In exemplary embodiments, at least one pocket 330 may be on the inside 314 of the support panel 310 and at least one pocket 330 may be on the outside 312 of the support panel 310.

Each pocket 330 is adapted to hold at least one support arm 340. In exemplary embodiments, the support arm 340 is generally flat elongated piece of material that has a degree of stiffness. In exemplary embodiments, the support arm 340 may be made of metal, alloy, composite, plastic, wood, combinations of the foregoing, or the like. In exemplary embodiments, each pocket 330 may be adapted to hold a plurality of support arms 340.

In exemplary embodiments, the pockets 330 may be spaced around the support panel 310 such that at least one support arm 340 is positioned on either side of the knee, such as in a first side pocket 342 and a second side pocket 344. In exemplary embodiments, at least one support arm 340 is positioned in rear pocket 346 proximate to the area behind the knee joint. In exemplary embodiments, several support arms 340 are positioned in rear pockets 346 to provide a wider bracing support area. In exemplary embodiments, the support arm or arms 340 that are behind the knee joint area may have a curved portion to accommodate the leg curvature. In exemplary embodiments, each support arm 340 is in its own pocket. In other exemplary embodiments, one pocket 330 may accommodate more than one support arm 340.

In use, a user slides a support arm 340 into a pocket 330. The user wraps the support panel 310 around the knee area so that the first and second side edges 316, 318 are positioned over the kneecap or slightly to one or both sides of the kneecap. The closure devices 322 of one row 320 of the first side edge 316 are secured to the mating closure devices 326 in one row 324 of the second side edge 318 to make the brace support 300 snug around the user's leg. The support arms 340 restrict bending of the knee.

The support arms 340 may be removed so that the support 300 can be washed or worn without the support arms 340.

While the methods, equipment and systems have been described in connection with specific embodiments, it is not intended that the scope be limited to the particular embodiments set forth, as the embodiments herein are intended in all respects to be illustrative rather than restrictive.

As used in the specification and the appended claims, the singular forms “a,” “an” and “the” include plural referents unless the context clearly dictates otherwise.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods, equipment and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods, equipment and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

It should further be noted that any patents, applications and publications referred to herein are incorporated by reference in their entirety.

What is claimed is:

1. A garment for supporting and maintaining at least one knee brace in position proximate to a knee, the knee brace comprising a pair of support arms connected to a hinge, the garment comprising:
a) a first leg panel having a thigh portion, a knee portion and a calf portion;
b) an upper outside pocket and a lower outside pocket, each pocket associated with the first leg panel, each pocket sized to accept at least a portion of a support arm;
c) an upper pocket and a lower inside pocket, each pocket associated with the first leg panel, each pocket sized to accept at least a portion of a support arm and,
d) a plurality of strap members, each strap member having a first end and a second end adjustably connectable by an adjustment connector, each strap member being associated with a portion of a pocket.

2. The garment of claim 1, further comprising a second leg panel and a waist panel.

3. The garment of claim 1, wherein the first leg panel further includes a zipper.

4. The garment of claim 1, wherein the second leg panel further includes a zipper.

5. The garment of claim 1, further comprising a reinforcement panel associated with the first leg panel knee portion.

6. The garment of claim 1, wherein at least a portion of the first leg panel comprises a compression fabric.

7. The garment of claim 1, wherein each strap member comprises a first strap segment and a second strap member.

8. The garment of claim 1, further comprising at least one reinforcement panel.

9. The garment of claim 1, further comprising at least one cushioning pad made of a cushioning material.

10. The garment of claim 2, wherein the second panel has
a) a thigh portion, a knee portion and a calf portion;
b) upper and lower outside pockets associated with the second leg panel, each pocket sized to accept at least a portion of a support arm;
c) upper and lower inside pockets associated with the second leg panel, each pocket sized to accept at least a portion of a support arm and,
d) a plurality of strap members, each strap member having a first end and a second end adjustably connectable by an adjustment connector, each strap member being associated with a portion of a pocket.

11. The garment of claim 1, wherein the first panel further comprises a bottom opening, rear area, side areas and front area, and further includes a seam extending generally vertically from an area proximate to the bottom opening in the rear area toward the knee area and curves proximate the knee portion toward one side area and further extends toward the thigh area.

12. A support garment for supporting and maintaining at least one knee brace in position proximate to a knee, each knee brace comprising a pair of support arms connected to a hinge, the garment comprising:
a) a first leg panel having
i) a thigh portion, a knee portion and a calf portion;
ii) upper and lower outside pockets associated with the first leg panel, each pocket sized to accept at least a portion of a support arm;
iii) upper and lower inside pockets associated with the first leg panel, each pocket sized to accept at least a portion of a support arm and,
iv) a plurality of strap members, each strap member having a first end and a second end adjustably connectable by an adjustment connector, each strap member being associated with a portion of a pocket.
b) a second leg panel having
i) a thigh portion, a knee portion and a calf portion;
ii) upper and lower outside pockets associated with the first leg panel, each pocket sized to accept at least a portion of a support arm;
iii) upper and lower inside pockets associated with the first leg panel, each pocket sized to accept at least a portion of a support arm and,
iv) a plurality of strap members, each strap member having a first end and a second end adjustably connectable by an adjustment connector, each strap member being associated with a portion of a pocket.

13. The support garment of claim 11, wherein the first panel further includes a bottom opening and a zipper extending from the first panel bottom opening toward the first panel thigh portion.

14. The support garment of claim 11, wherein the second panel further includes a bottom opening and a zipper extending from the second panel bottom opening toward the second panel thigh portion.

15. A knee brace support, comprising:
a) a fabric panel having a first side edge, a second side edge, a top edge, and a bottom edge;
b) at least one row of first closure devices associated with an area proximate to the first side edge;
c) at least one row of second closure devices associated with an area proximate to the second side edge, the second closure devices adapted to fasten to the first closure devices; and,
d) at least one pocket associated with the fabric panel, each pocket having an opening.

16. The knee brace support of claim 15, further comprising at least one support arm removably retained in each pocket.
